(19) United States
(12) Patent Application Publication
(10) Pub. No.: US 2014/0302536 A1
(43) Pub. Date: Oct. 9, 2014

(54) DIAGNOSIS OF PULMONARY AND/OR CARDIOVASCULAR DISEASE

(71) Applicant: Critical Care Diagnostics, Inc., San Diego, CA (US)

(72) Inventors: James V. Snider, San Diego, CA (US); Sven Jacobson, New York, NY (US)

(21) Appl. No.: 14/312,221

(22) Filed: Jun. 23, 2014

Related U.S. Application Data
(63) Continuation of application No. 12/299,282, filed on Nov. 16, 2009.
(60) Provisional application No. 60/797,285, filed on May 2, 2006.

(51) Int. Cl. G01N 33/53; G01N 33/68; C12Q 1/68

(52) U.S. Cl. CPC G01N 33/53; C12Q 1/6883; G01N 33/6893
USPC 435/7.92; 436/501; 436/94; 435/15; 435/4

(57) ABSTRACT

Described are methods and kits for determining the likelihood of the presence of cardiovascular disease (CVD) or pulmonary disease (PD) in a subject using ST2/Interleukin 1 Receptor Like 1 (IL1RL1) and/or Interleukin 33 (IL-33), and a biomarker for CVD, e.g., a natriuretic peptide, e.g., brain natriuretic peptide (BNP), prohormone BNP (proBNP), N-Terminal proBNP (NT-proBNP), atrial natriuretic peptide (ANP), proANP, or NT-proANP.

### CROSS-REFERENCE TO RELATED APPLICATIONS

This application is a continuation application of U.S. patent application Ser. No. 12/299,282, filed Oct. 31, 2008, which is a U.S. national stage under 35 U.S.C. §371 of International Application Number PCT/US2007/068024, filed on May 2, 2007, which claims the benefit under 35 U.S.C. §119(e) to U.S. Provisional Patent Application Ser. No. 60/797,285, filed on May 2, 2006, the entire contents of which are hereby incorporated by reference.

### TECHNICAL FIELD

This invention relates to methods for determining the likelihood of the presence of cardiovascular disease (CVD) or pulmonary disease (PD) in a subject using ST2 (which is also known as Interleukin 1 Receptor Like 1 (IL1RL1)) and/or Interleukin 33 (IL-33), and a diagnostic biomarker, e.g., a natriuretic peptide, e.g., brain natriuretic peptide (BNP), prohormone BNP (proBNP), N-terminal proBNP (NT-proBNP), atrial natriuretic peptide (ANP), proANP, or NT-proANP.

### BACKGROUND

Non-specific symptoms such as dyspnea and chest pain are a common problem in the outpatient primary care setting. Establishing a diagnosis can be challenging because the differential diagnosis can include multiple diagnostic categories, including cardiovascular and pulmonary diseases. Underlying disorders can range from relatively benign conditions (e.g., hyperventilation) to more serious and even life-threatening diseases (e.g., pulmonary embolism or heart failure), which are best addressed in an emergency department. Timely assessment, accurate diagnosis, and initiation of appropriate therapy play a critical role in optimizing treatment and patient recovery.

### SUMMARY

The present invention includes methods for determining the likelihood of the presence of cardiovascular disease (CVD) or pulmonary disease (PD) in a subject. The methods use ST2 (IL1RL1) and/or Interleukin-33 (IL-33), in combination with other biomarkers including the natriuretic peptides (NPs).

In one aspect, the invention provides methods for determining the likelihood of the presence of a cardiovascular disease or a pulmonary disease in a subject. The methods include optionally obtaining a biological sample from the subject; determining a level of a first biomarker selected from the group consisting of ST2 and/or IL-33 in such a sample; determining a level of a second biomarker for a cardiovascular disease (CVD) in the sample; and comparing the levels of the first and second biomarkers in the sample to reference levels. The levels of the biomarkers in the sample as compared to the reference levels is correlated with (i.e., is statistically correlated with) the likelihood that the subject has a pulmonary disease or CVD. In some embodiments, the subject has a non-specific symptom, e.g., dyspnea or chest pain, that suggests a diagnosis of a pulmonary disease or CVD, and the biomarker levels indicate which of the two diseases, if any, the subject has.

The CVD biomarker is a biomarker that is diagnostic of a cardiovascular condition. In some embodiments, the CVD biomarker is a natriuretic peptide (NP), e.g., brain natriuretic peptide (BNP), prohormone BNP (proBNP), N-terminal proBNP (NT-proBNP), atrial natriuretic peptide (ANP), proANP, or NT-proANP. In some embodiments, the CVD biomarker is BNP.

In some embodiments of the methods described herein, the biological sample comprises blood, serum, plasma, urine, or body tissue. In some embodiments, the sample is a serum sample.

In some embodiments of the methods described herein, the reference levels represent levels in a subject who does not have a CVD or a PD. In some embodiments, the reference levels represent levels in a subject who has a CVD. In some embodiments, the reference levels represent levels in a subject who has a PD, or both a PD and a CVD.

Determining a level of a biomarker in the sample can include contacting a binding composition to the sample, wherein the binding composition specifically binds to the biomarker, and measuring or determining the specific binding of the binding composition to the sample. Suitable binding compositions include antibodies that bind specifically to a biomarker polypeptide and oligonucleotide probes that bind specifically to a polynucleotide encoding a biomarker.

In some embodiments, the subject has dyspnea and the CVD diagnosed by a method described herein is congestive heart failure, coronary artery disease (CAD), arrhythmia, pericarditis, acute myocardial infarction, or anemia.

In some embodiments, the subject has dyspnea and the pulmonary disease diagnosed by methods described herein is chronic obstructive pulmonary disease (COPD), asthma, pneumonia, pneumothorax, pulmonary embolism, pleural effusion, metastatic disease, pulmonary edema, gastrooesophageal reflux disease with aspiration, or restrictive lung disease.

In some embodiments, the methods described herein also include determining a level in the sample of one or more other additional biomarkers, e.g., biomarkers selected from the group consisting of troponin, myoglobin, creatine kinase MB (CK-MB), ischemia-modified albumin (IMA), Interleukin-6 (IL-6), C-reactive protein (CRP), creatinine, D-dimers, blood urea nitrogen (BUN), liver function enzymes, albumin, and bacterial endotoxin.

In an additional aspect, the invention features kits for diagnosing pulmonary diseases. The kits include one or more separate antibodies that each specifically bind to a biomarker used in the methods described herein, and/or an oligonucleotide probe that specifically binds to a nucleic acid encoding said biomarkers, and instructions for use in a method described herein.

Also described herein are kits for determining the likelihood of the presence of cardiovascular disease (CVD) or pulmonary disease (PD) in a subject. The kits include (i) one or both of a binding composition that specifically binds to IL-33 and a binding composition that specifically binds to ST2, (ii) a binding composition binds to a biomarker for CVD, and (iii) instructions for use in a method of determining the likelihood of the presence of CVD or PD in a subject described herein. In some embodiments, the IL-33 and/or ST2 and the CVD biomarker comprise polypeptides, and the binding composition includes an antibody or antigen binding fragment thereof that binds specifically to each of the polypeptides. In some embodiments, the IL-33 and/or ST2 and the CVD biomarker comprises a nucleic acid or a nucleic acid probe that binds specifically to each of the nucleic acids.

A “cardiovascular disease,” as used herein, refers to a disorder of the heart and blood vessels, and includes disorders of the arteries, veins, arterioles, venules, and capillaries. Cardiovascular diseases diagnosed by a method described herein can include congestive heart failure (HF), coronary artery disease (CAD), arrhythmia, pericarditis, and acute myocardial infarction (MI).

A “pulmonary disease,” as used herein, refers to a disorder of the lungs. Pulmonary diseases diagnosed by methods described herein can include chronic obstructive pulmonary disease (COPD), asthma, pneumonia, pneumothorax, pulmonary embolism, pleural effusion, metastatic disease, pulmonary edema, gastrooesophageal reflux disease with aspiration, and/or restrictive lung disease.

“Upregulated,” as used herein, refers to increased expression of a gene and/or its encoded polypeptide. “Increased expression” refers to increasing (i.e., to a detectable extent) replication, transcription, and/or translation of a gene, e.g., ST2, since upregulation of any of these processes results in an increase in concentration/amount of the polypeptide encoded by the gene. Conversely, “downregulation,” or “decreased expression” as used herein, refers to reduced replication, transcription, and/or translation of the gene and/or its encoded polypeptide. The upregulation or downregulation of gene expression can be directly determined by detecting an increase or decrease, respectively, in the level of mRNA for the gene, or the level of protein expression of the gene-encoded polypeptide, using any suitable means known to the art, such as nucleic acid hybridization or antibody detection methods, respectively, and in comparison to controls. “Expression,” as used herein, refers to nucleic acid and/or polypeptide expression.

As used herein, a “subject” is a mammal, e.g., a human or non-human mammal In general, human nucleic acids and polypeptides, or nucleic acid molecules or polypeptides synthesized or generated to have sequences based on a corresponding human nucleic acid or polypeptide sequence, are preferred for use in diagnosing human subjects.

As used herein, a “sample” includes one or more of blood, serum, plasma, urine, and body tissue. In some embodiments, a sample is a serum or blood sample.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a table illustrating the effect of COPD history on ST2 concentrations in subjects with/without acute HF.

FIG. 2 is a box graph illustrating levels of ST2 in subjects with COPD/Asthma and/or HF.

### DETAILED DESCRIPTION

Accurate diagnosis of subjects presenting with non-specific symptoms can be difficult, as the symptoms can have very different etiology. For example, subjects with dyspnea (shortness of breath, or uncomfortable breathing) may be suffering from a pulmonary disease or a cardiovascular disease, both, or neither.

General Methodology

In general, the methods described herein include evaluating levels of a first biomarker (ST2 and/or IL-33) and a second, CVD biomarker (e.g., a natriuretic peptide (NP)) in a biological sample (e.g., a blood, serum, plasma, urine, or body tissue sample) from a subject, e.g., a mammal, e.g., a human. These levels can provide diagnostic information, e.g., indicating whether the subject has a pulmonary disease (PD) or a cardiovascular disease (CVD), as described herein. In some embodiments, the second biomarker is a NP selected from the group consisting of brain natriuretic peptide (BNP), proBNP, NT-proBNP, atrial natriuretic peptide (ANP), proANP, or NT-proANP.

In one example, diagnosis of CVD versus PD can be made by referring to Table 1.

TABLE 1
Diagnosis of CVD vs PD

| | Low ST2 | |
|---|---|---|
| | (e.g., <0.20 ng/ | High ST2 |
| | mL serum) | (e.g., ≧0.20 ng/mL serum) |
| Low BNP | low probability of | probable PD |
| (<100 pg/mL) | either CVD or PD | |
| Moderate BNP | possible CVD | possible PD |
| (100-500 pg/ml) | | |
| High BNP | probable CVD | highly probable CVD |
| (>500 pg/ml) | | |

Thus, for a subject who has both low levels of a CVD biomarker, e.g., BNP, and low levels of ST2, there is a low probability of the presence of either CVD or PD. Low levels of the CVD biomarker and high levels of ST2 indicate a greater likelihood of PD than CVD.

For a subject who has moderate levels of BNP, low levels of ST2 indicate that the presence of CVD is possible (and more likely than PD), while high levels of ST2 indicate that the presence of PD is possible (and more likely than CVD).

Finally, for a subject who has high levels of a CVD biomarker, low levels of ST2 indicate that CVD is probable (and more likely than PD), and high levels of ST2 indicate that CVD is highly probable (and more likely than PD).

Using the methods described herein, diagnoses can be ruled in or ruled out for a given subject, allowing a care giver to focus diagnostic efforts, and thus therapeutic efforts, appropriately. Although it is possible that both etiologies may exist simultaneously in a subject, at any given point in time (and particularly in an acute care situation such as presentation with dyspnea) it is likely one etiology would be more “dominant” than the other, and thus would be the primary target for treatment.

In some embodiments, the levels of the biomarkers are determined once, e.g., at presentation. In some embodiments, the levels of the biomarkers are determined at any one or more of 1, 2, 3, 4, 5, 6, 7, 8, 12, 18, and/or 24 hours, and/or at 1-7 days or longer, after the onset of symptoms.

In embodiments where the levels of the biomarkers are determined more than once, the highest level or an average can be used, or the change in levels can be determined and used. Levels of the biomarkers can also be determined multiple times to evaluate a subject's response to a treatment. For example, levels, e.g., of IL-33 and/or ST2, that are taken after administration of a treatment, e.g., one or more doses or rounds of a treatment, can be compared to levels taken before the treatment was initiated, e.g., baseline levels. The change in levels would indicate whether the treatment was effective; e.g., a reduction in levels would indicate that the treatment was effective.

Evaluating levels of the biomarker in a subject typically includes obtaining a biological sample, e.g., serum or blood, from the subject. Levels of the biomarkers in the sample can be determined by measuring levels of biomarker polypeptides in the sample, using methods known in the art and/or described herein, e.g., immunoassays such as enzyme-linked immunosorbent assays (ELISA). Alternatively, levels of mRNA encoding the biomarkers can be measured, again using methods known in the art and/or described herein, e.g., by quantitative PCR or Northern blotting analysis.

For example, a method as described herein, e.g., for differential diagnosis of pulmonary disease, can include contacting a sample from a subject, e.g., a sample including blood, serum, plasma, urine, or body tissue from the subject, with a binding composition (e.g., an antibody or oligonucleotide probe) that specifically binds to a polypeptide or nucleic acid of the biomarkers as described herein. The methods can also include contacting a sample from a control subject, normal subject, or normal tissue or fluid from the test subject, with the binding composition, e.g., to provide a reference or control. Moreover, the method can additionally include comparing the specific binding of the composition to the test subject with the specific binding of the composition to the normal subject, control subject, or normal tissue or fluid from the test subject. Expression or activity of biomarkers in a test sample or test subject can also be compared with that in a control sample or control subject. A control sample can include, e.g., a sample from a non-affected subject, or a subject who has a known condition, e.g., a pulmonary disease or a cardiovascular disease. Expression or activity from a control subject or control sample can be provided as a predetermined value, e.g., acquired from a statistically appropriate group of control subjects.

An antibody that “binds specifically to” an antigen, binds preferentially to the antigen in a sample containing other proteins. The term “antibody” as used herein refers to an immunoglobulin molecule or immunologically active portion thereof, i.e., an antigen-binding portion. Examples of immunologically active portions of immunoglobulin molecules include F(ab) and F(ab′)₂ fragments which can be generated by treating the antibody with an enzyme such as pepsin. The antibody can be polyclonal, monoclonal, recombinant, e.g., a chimeric or humanized, fully human, non-human, e.g., murine, monospecific, or single chain antibody. In some embodiments it has effector function and can fix complement.

An “oligonucleotide probe” (also referred to simply as a “probe”) is a nucleic acid that is at least 10, and less than 200 (typically less than about 100 or 50) base pairs in length. A probe that “binds specifically to” a target nucleic acid hybridizes to the target under high stringency conditions. As used herein, the term “hybridizes under high stringency conditions” describes conditions for hybridization and washing. As used herein, high stringency conditions are 0.5 M sodium phosphate, 7% SDS at 65° C., followed by one or more washes at 0.2×SSC, 1% SDS at 65° C. Methods for performing nucleic acid hybridization assays are known to those skilled in the art and can be found in Current Protocols in Molecular Biology, John Wiley & Sons, N.Y. (1989), 6.3.1-6.3.6.

Detection can be facilitated by coupling (i.e., physically linking) the antibody or probe to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, quantum dots, or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Diagnostic assays can be used with biological matrices such as live cells, cell extracts, cell lysates, fixed cells, cell cultures, bodily fluids, or forensic samples. Conjugated antibodies useful for diagnostic or kit purposes, include antibodies coupled to dyes, isotopes, enzymes, and metals, see, e.g., Le Doussal et al., New Engl. J. Med. 146:169-175 (1991); Gibellini et al., J. Immunol. 160:3891-3898 (1998); Hsing and Bishop, New Engl. J. Med. 162:2804-2811 (1999); Everts et al., New Engl. J. Med. 168:883-889 (2002). Various assay formats exist, such as radioimmunoassays (RIA), ELISA, and lab on a chip (U.S. Pat. Nos. 6,176,962 and 6,517,234).

Known techniques in biochemistry and molecular biology can be used in the methods described herein (see, e.g., Maniatis et al., Molecular Cloning, A Laboratory Manual, Cold Spring Harbor Laboratory Press, Cold Spring Harbor, N.Y. (1982); Sambrook and Russell, Molecular Cloning, 3^{rd} ed., Cold Spring Harbor Laboratory Press, Cold Spring Harbor, N.Y. (2001); Wu, Recombinant DNA, Vol. 217, Academic Press, San Diego, Calif. (1993); and Ausbel et al., Current Protocols in Molecular Biology, Vols. 1-4, John Wiley and Sons, Inc. New York, N.Y. (2001)).

Once a level of a biomarker has been determined, the level can be compared to a reference level. In some embodiments, the reference level will represent a threshold level, above which the subject can be diagnosed with CVD or PD disease. The reference level chosen may depend on the methodology used to measure the levels of the biomarkers. In some embodiments, the reference level is a range of levels.

In some embodiments, both levels of ST2 and IL-33 are determined, and the information from the comparison of both biomarkers with their respective reference levels provides cumulative information regarding the presence of pulmonary disease in the subject, and/or the presence of a severe disease in the subject. In some embodiments, the ratio of ST2 to IL-33 may be determined, and the ratio compared to a reference ratio that represents a threshold ratio above which the subject has CVD or PD, e.g., as shown in Table 1.

ST2/Interleukin 1 Receptor-Like 1 (IL1RL1)

The ST2 gene is a member of the interleukin-1 receptor family, whose protein product exists both as a trans-membrane form, as well as a soluble receptor that is detectable in serum (Kieser et al., FEBS Lett. 372(2-3):189-93 (1995); Kumar et al., J. Biol. Chem. 270(46):27905-13 (1995); Yanagisawa et al., FEBS Lett. 302(1):51-3 (1992); Kuroiwa et al., Hybridoma 19(2):151-9 (2000)). ST2 was recently described to be markedly up-regulated in an experimental model of heart failure (Weinberg et al., Circulation 106(23):2961-6 (2002)), and preliminary results suggest that ST2 concentrations may be elevated in those with chronic severe HF (Weinberg et al., Circulation 107(5):721-6 (2003)) as well as in those with acute myocardial infarction (MI) (Shimpo et al., Circulation 109(18):2186-90 (2004)).

The transmembrane form of ST2 is thought to play a role in modulating responses of T helper type 2 cells (Lohning et al., Proc. Natl. Acad. Sci. U.S.A. 95(12):6930-5 (1998); Schmitz et al., Immunity 23(5):479-90 (2005)), and may play a role in development of tolerance in states of severe or chronic inflammation (Brint et al., Nat. Immunol. 5(4):373-9 (2004)), while the soluble form of ST2 is up-regulated in growth stimulated fibroblasts (Yanagisawa et al., 1992, supra). Experimental data suggest that the ST2 gene is markedly up-regulated in states of myocyte stretch (Weinberg et al., 2002, supra) in a manner analogous to the induction of the BNP gene (Bruneau et al., Cardiovasc. Res. 28(10):1519-25 (1994)).

Tominaga, FEBS Lett. 258:301-304 (1989), isolated murine genes that were specifically expressed by growth stimulation in BALB/c-3T3 cells; they termed one of these genes St2 (for Growth Stimulation-Expressed Gene 2). The St2 gene encodes two protein products: ST2 (IL1RL1), which is a soluble secreted form; and ST2L, a transmembrane receptor form that is very similar to the interleukin-1 receptors. The HUGO Nomenclature Committee designated the human homolog, the cloning of which was described in Tominaga et al., Biochim. Biophys. Acta. 1171:215-218 (1992), as Interleukin 1 Receptor-Like 1 (IL1RL1). The two terms (ST2 and IL1RL1) are used interchangeably herein.

The mRNA sequence of the shorter, soluble isoform of human ST2 can be found at GenBank Acc. No. NM_{—}003856.2, and the polypeptide sequence is at GenBank Acc. No. NP_{—}003847.2; the mRNA sequence for the longer form of human ST2 is at GenBank Acc. No. NM_{—}016232.4; the polypeptide sequence is at GenBank Acc. No. NP_{—}057316.3. Additional information is available in the public databases at GeneID: 9173, MIM ID #601203, and UniGene No. Hs.66. In general, in the methods described herein, the soluble form of ST2 polypeptide is measured.

Methods for detecting and measuring ST2 are known in the art, e.g., as described in U.S. Pat. Pub. Nos. 2003/0124624, 2004/0048286 and 2005/0130136, the entire contents of which are incorporated herein by reference. Kits for measuring ST2 polypeptide are also commercially available, e.g., the ST2 ELISA Kit manufactured by Medical & Biological Laboratories Co., Ltd. (MBL International Corp., Woburn, Mass.), no. 7638. In addition, devices for measuring ST2 and other biomarkers are described in U.S. Pat. Pub. No. 2005/0250156.

In some embodiments, the level of ST2 is determined once, e.g., at presentation. In some embodiments, the level of ST2 is determined at one or more of 2, 4, 6, 8, 12, 18, and/or 24 hours, and/or 1-7 days after the onset of symptoms.

In some embodiments, the level of ST2 is determined more than once; in that case, the higher measurement can be used. In embodiments where the level of ST2 is determined more that once, the highest level can be used, or the change in levels can be determined and used. Levels of ST2 can also be determined multiple times to evaluate a subject's response to a treatment. For example, a level of ST2 taken after administration of a treatment, e.g., one or more doses or rounds of a treatment, can be compared to levels of ST2 before the treatment was initiated, e.g., a baseline level. The change in ST2 levels would indicate whether the treatment was effective; e.g., a reduction in ST2 levels would indicate that the treatment was effective.

In some embodiments, the methods include determining the identity of the nucleotide sequence at RefSNP ID: rs1041973.

Interleukin-33 (IL-33)

IL-33 was recently identified as the ligand for ST2, and the presence of increased levels of IL-33 in various inflammatory disorders has been described (see Schmitz et al., Immunity 23(5):479-90 (2005); U.S. Pat. Pub. No. 2005/0203046). In the methods described herein, ST2 can be measured in addition to IL-33. The ratio of ST2 to IL-33 can also be determined, as can ratios of bound complexes to bound and/.

IL-33 protein is expressed as an inactive molecule, pre-IL-33, that is activated after cleavage by Caspase I resulting in the active IL-33 peptide as well as the cleavage peptide product, pro-IL-33. Therefore, the methods described herein can include measuring one, two, or all three of mature IL-33, pre-IL-33, and/or pro-IL-33, all of which are included in the term “IL-33.”

The nucleic acid sequence of IL-33 can be found at GenBank Acc. No. NM_{—}033439.2, and the polypeptide sequence is at GenBank Acc. No. NP_{—}254274.1. Additional information is available in the public databases at GeneID: 90865, MIM ID #*608678, and UniGene No. Hs.348390. IL-33 is also known as Chromosome 9 Open Reading Frame 26 (C9ORF26); Nuclear Factor from High Endothelial Venules (NFHEV); and Interleukin 33. See also Baekkevold et al., Am. J. Path. 163: 69-79 (2003).

Methods for measuring levels of IL-33 polypeptide and nucleic acid are known in the art, see, e.g., Schmitz et al., Immunity 23(5):479-90 (2005); U.S. Pat. Pub. No. 2005/0203046.

CVD Biomarkers

The methods described herein include measuring levels of CVD biomarkers in addition to IL1RL1 (ST2) and/or IL-33. Suitable biomarkers for CVD include troponin, NT-proBNP, BNP, NT-proANP, and ANP.

In some embodiments, the CVD diagnostic biomarker is B-type natriuretic peptide (BNP), a marker of hemodynamic stress characteristic of heart failure. Levels of BNP can be determined, e.g., in whole blood or serum, using standard methodology. For example, a number of assay kits are commercially available, e.g., the Triage BNP Test (Biosite, Inc., San Diego, Calif.), a point-of-care assay that whole blood or plasma and produces results in about 15 minutes; a chemiluminescent sandwich immunoassay (Bayer HealthCare Diagnostics, Tarrytown, N.Y.) for BNP that is run on the ADVIA Centaur and ACS:180 platforms; a microparticle-based immunoassay (Abbott Laboratories, Abbott Park, Ill.) for BNP that is run on the AxSYM platform; and a chemiluminescent immuno-enzymatic assay (Biosite, Inc., San Diego, Calif.) for BNP that is run on the following Beckman Coulter platforms: Access, Access 2, Synchron LXI and the UniCel DXI. An electrochemiluminescent assay (Roche Diagnostics, Indianapolis, Ind.) available for measuring NT-proBNP.

The reference ranges for BNP and NTproBNP vary depending on a number of factors. The following ranges are for use where BNP levels are measured using an ELISA-type method, and one of skill in the art will be able to determine what levels obtained using other methods are equivalent. If the BNP level is >500 pg/mL, then HF is highly likely. Levels of BNP of 100-500 pg/mL are often described as a “grey zone,” in which diagnosis is less certain. In lean subjects, if the BNP is <100 pg/mL, then HF is unlikely, however, obesity influences the expression of BNP in chronic HF (Mehra et al., J Am Coll Cardiol. 43(9):1590-1595 (2004)), so levels of <100 pg/mL do not rule out heart failure in obese subjects (Silver et al., Cong. Heart Fail. 10(5 suppl. 3):1-30 (2004)).

Other Biomarkers

In some embodiments, the methods also include measuring levels of other biomarkers, e.g., one or more of: troponin, creatine kinase MB (CK-MB), Myoglobin (Myo), ischemia-modified albumin (IMA), Interleukin-6 (IL-6), C-reactive protein (CRP), creatinine, D-dimers, blood urea nitrogen (BUN), liver function enzymes, albumin, and/or bacterial endotoxin. Methods for measuring these biomarkers are known in the art, see, e.g., U.S. Pat. Pub. Nos. 2004/0048286 and 2005/0130136 to Lee et al.; Dhalla et al., Mol. Cell Biochem. 87:85-92 (1989); Moe et al., Am. Heart J. 139:587-95 (2000), the entire contents of which are incorporated herein by reference.

Kits

Also included herein are kits that include a reagent comprising a binding composition for the detection of one or more of the IL-33 or ST2 polypeptide(s) or nucleic acid, e.g., an anti-IL-33 or ST2 antibody (i.e., an antibody or antigen binding fragment thereof that binds specifically to IL-33 or ST2), or a nucleic acid probe complementary to all or part of the IL-33 or ST2 nucleic acid), as well as a reagent comprising a binding composition for the detection of one or more of a CVD biomarker, e.g., a CVD biomarker polypeptide or a nucleic acid encoding a CVD biomarker, and instructions for use in a method described herein. A control can also be included, e.g., an epitope of IL-33 or ST2, and of the CVD biomarker.

Kits are generally comprised of the following major elements: packaging, reagents comprising binding compositions as described above, optionally a control, and instructions. Packaging may be a box-like structure for holding a vial (or number of vials) containing said binding compositions, a vial (or number of vials) containing a control, and instructions for use in a method described herein. Individuals skilled in the art can readily modify the packaging to suit individual needs.

As one example, the kit may contain an antibody or antigen binding fragment thereof that binds specifically to ST2 (or IL-33), and an antibody or antigen binding fragment thereof that binds specifically to a CVD biomarker, e.g., BNP, proBNP, NT-proBNP, ANP, proANP, or NT-proANP.

In some embodiments, other methods of detection can be used, e.g., colorimetric assays, radioimmunoassays, or chemiluminescent assays. Sandwich assays can be used as well, e.g., using two monoclonal antibodies, one labelled with iodine 125 and the other adsorbed onto beads, e.g., as used in the IRMA-BNP2 kit from CISBIO International (France) and the ShionoRIA BNP or ANP kits (SHIONOGI USA Inc.).

For example, the kit can be designed for use in a assay is a chemiluminescent microparticle immunoassay (CMIA), such as the ARCHITECT assays from Abbot Diagnostics (Abbott Park, Ill.), and thus can contain paramagnetic microparticles coated with anti-BNP antibodies, and paramagnetic microparticles coated with anti-ST2 antibodies. These microparticles are contacted with a sample, and the BNP and ST2 present in the sample bind to the coated microparticles. Optionally, the sample can be split into at least two aliquots, and each type of microparticle can be contacted with a separate aliquot. After washing, anti-BNP and anti-ST2 acridinium-labeled conjugate can be added to create a reaction mixture in the second step. Following another wash cycle pre-trigger and trigger solutions are added to the reaction mixture. The resulting chemiluminescent reaction is measured, e.g., using the ARCHITECT i System optics (Abbot Diagnostics, Abbott Park, Ill.). A direct relationship exists between the amount of BNP or ST2 in the sample and the chemiluminescence detected.

### EXAMPLES

The invention is further described in the following examples, which do not limit the scope of the invention described in the claims.

### Example 1

### Detection and Measurement of ST2 in the Serum

This example uses the ST2 ELISA Kit manufactured by Medical & Biological Laboratories Co., Ltd. (MBL International Corp., Woburn, Mass.), no. 7638. This kit is a sandwich ELISA assay utilizing monoclonal antibodies for both capture and detection. This procedure is intended to analyze a full plate of samples assayed in replicates at a 1:3 dilution factor and closely follows the manufacturers' protocol. Kits should be stored at 4° C. until use. The procedure described in this example is optimized for human serum or plasma collected in citrate or EDTA anticoagulant tubes. Plasma collected in heparin anticoagulant tubes should not be used in this assay as heparin binds ST2 and inhibits measurement by this ELISA protocol. Plasma or serum samples may be used fresh or stored frozen. This assay is not adversely affected by up to 3 freeze and thaw cycles of plasma samples.

Reagents should be prepared fresh from a new kit immediately before performing the assays. Allow the kit to equilibrate to room temperature prior to use. Reagents not explicitly discussed below are provided by the manufacturer ready to use.
1. Wash solution—wash solution is provided by the manufacturer as a 10× concentrate solution. To make 1 liter of wash solution dilute 100 ml of the 10× concentrate provided with 900 ml of distilled water.
2. Detector solution—the detector solution is prepared by diluting the detector concentrate 1:101 with the detector diluent. For a full 96 well plate of samples 10 ml of detector solution is required. To prepare 10 ml of detector solution use a pipette to transfer 10 ml of the blue colored detector diluent to a 15 ml orange top polypropylene tube. Ad 100 μl of the detector concentrate to this volume of detector diluent.
   a. NOTE: this reagent should be prepared during the first assay incubation step.
3. Calibrator stock—reconstitute the calibrator protein by dissolving the lyophilized protein in the amount of distilled water defined by the manufacturer for this manufacturing lot to yield a stock solution of 8 ng/ml. This volume specification is included in the product insert.

Preparation of Standards and Samples:
All of the following should be prepared in labeled 1.5 ml polypropylene tubes to be transferred to the assay plate with the P200 pipetter.

Standards:

The standard curve is prepared by making 2 fold serial dilutions of the 8 ng/ml stock solution.
1. Using a P1000 pipette transfer 250 μl of Assay Diluent to 8 1.5 ml polypropylene tubes labeled S1-S8
2. Using the same P1000 pipette transfer 250 μl of the 8 ng/ml Calibrator stock solution to tube S1. This tube is now 4 ng/ml calibrator protein.
   a. Mix thoroughly by gently pipetting 3 times being careful not to create bubbles.
3. Using the same P1000 pipette and a fresh tip for each of the following transfer 250 μl of the reagent in tube S1 to tube S2, repeat the mixing.
4. Repeat step 3 for S2 to S3, S3 to S4, S4 to S5, S5 to S6 and S6 to S7. S8 will be the reagent blank so do not transfer the calibrant protein to this well.
   a. Tubes S1-S6 and S8 will now have 250 μl of reagent and tube S7 will have 450 μl

Samples:

The plate is set up so that each sample is analyzed as a 1:3 dilution in duplicate.
1. Label a 1.5 ml polypropylene tube for each sample.
2. Using the P200 pipette transfer 160 μl of Assay Diluent to each tube.
3. Using a P200 pipette transfer 80 μl of serum or plasma from sample 1 to tube 1. Mix carefully by pipetting 3 times without making bubbles.
4. Continue transferring samples to the sample tubes by repeating step 2 for each sample.

Procedure:
1. Use the P200 pipette transfer the standards and diluted serum samples quickly to the 96 well assay plate. An exemplary layout is shown below in Table 2.
   a. Set the P200 pipette for 100 μl
   b. Transfer 100 μl of the standard curve dilutions to each of columns 1 & 2 in the assay plate
   c. Transfer 100 μl of each of the serum samples to the assay plate in exactly the same positions as shown in the plate map below.
2. Cover the assay plate with the provided shield and incubate at room temperature for 60 minutes.
3. Using the plate autowasher wash the plate 4 times.
4. Detector: using the 8 channel multichannel pipette transfer 100 μl of the detector solution to each well and incubate at room temperature for 60 minutes.
   a. NOTE: this reagent was to be prepared during the first incubation step.
   b. NOTE: use a disposable reagent vessel for this reagent addition. ALWAYS use a fresh disposable reagent vessel for each reagent. It is not necessary to change pipette tips during this step.
5. Wash the plate as in step 3
6. Substrate: using the 8 channel multichannel pipette transfer 100 μl of the Substrate to each well and incubate at room temperature for 30 minutes.
   a. The Substrate reagent is provided ready to use by the manufacturer.
7. Stop: at the completion of the Substrate incubation using the 8 channel multichannel pipette transfer 100 μl of the Stop solution to each well.
   a. The Stop Solution reagent is provided ready to use by the manufacturer.
8. Read the plate at 450 nm with background correction at 620 nm.
   a. The plate should be read within 30 minutes after stopping the reaction.
9. Enter the absorbance readings in the provided spreadsheet for analysis.

TABLE 2
Map of Exemplary 96 Well Assay Plate

| | 1 | 2 | 3 | 4 | 5 | 6 | 7 | 8 | 9 | 10 | 11 | 12 |
|---|---|---|---|---|---|---|---|---|---|---|---|---|
| A | 4.0 | | 1 | 1 | 9 | 9 | 17 | 17 | 25 | 25 | 33 | 33 |
| B | 2.0 | | 2 | 2 | 10 | 10 | 18 | 18 | 26 | 26 | 34 | 34 |
| C | 1.0 | | 3 | 3 | 11 | 11 | 19 | 19 | 27 | 27 | 35 | 35 |
| D | 0.5 | | 4 | 4 | 12 | 12 | 20 | 20 | 28 | 28 | 36 | 36 |
| E | 0.25 | | 5 | 5 | 13 | 13 | 21 | 21 | 29 | 29 | 37 | 37 |
| F | 0.125 | | 6 | 6 | 14 | 14 | 22 | 22 | 30 | 30 | 38 | 38 |
| G | 0.0625 | | 7 | 7 | 15 | 15 | 23 | 23 | 31 | 31 | 39 | 39 |
| H | 0.0 | | 8 | 8 | 16 | 16 | 24 | 24 | 32 | 32 | 40 | 40 |

### Example 2

### Detection and Measurement of IL-33 in the Serum

A blood sample is collected from a subject, and serum is prepared from the sample using standard methods. A labeled monoclonal antibody to IL-33 (e.g., as described U.S. Pat. App. Pub. No. 2005/0203046, incorporated herein by reference in its entirety) is added to the sample and incubated for a sufficient amount of time for binding to occur. The antibody/IL-33 complexes are then detected using standard methods, and the amount of IL-33 present is quantified. Levels of IL-33 are expected to correlate with disease in a manner similar to that of ST2, as described herein.

### Example 3

### COPD and ST2 Concentrations

The effect of COPD history on ST2 concentrations in subjects with/without acute HF was evaluated in subjects from the PRIDE study.

600 breathless subjects were enrolled in the PRIDE study, to analyze the utility of NT-proBNP for diagnosis and prognosis of acute heart failure (HF). At enrollment, a blinded sample of blood was obtained, processed and frozen at −80° C. For the purposes of ST2 analysis, an aliquot of citrated blood was thawed (second freeze-thaw cycle) and analyzed for concentration of ST2 protein as described in Example 1.

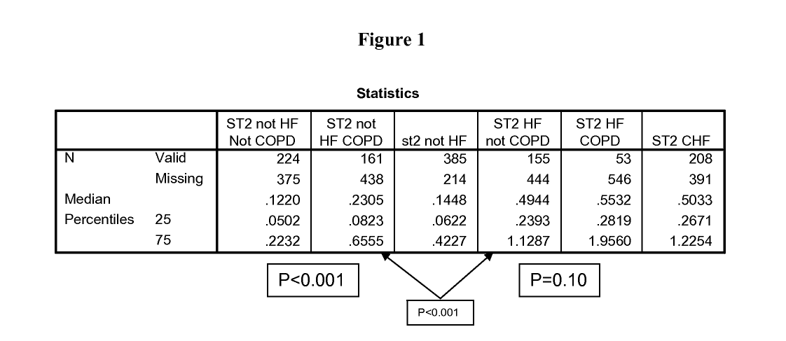

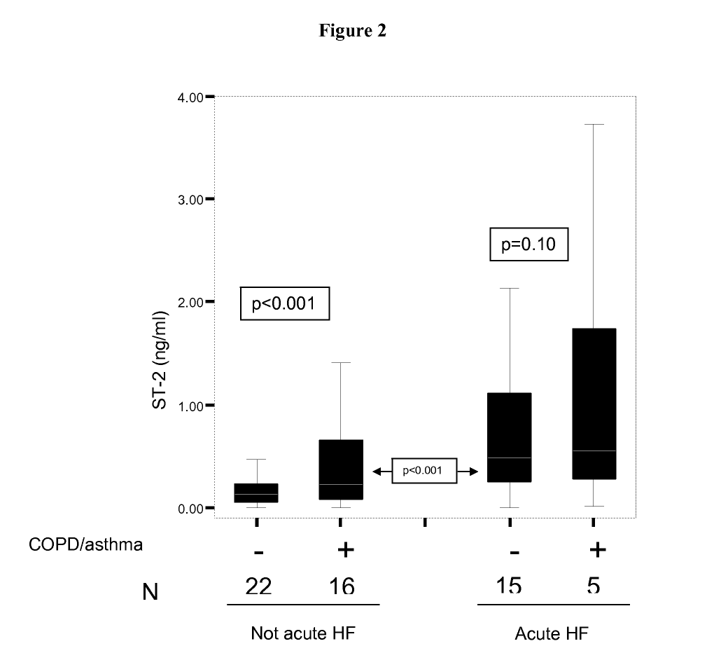

The results are shown in FIGS. 1-2, and demonstrate that elevated ST2 levels, e.g., above 0.2 ng/ml of serum when determined as described in Example 1, can be used to predict the likelihood of the presence of pulmonary disease, e.g., in subjects without acute decompensated heart failure, e.g., with low or moderate BNP levels.

### Example 4

### Elevated ST2 Concentrations in Patients without Heart Failure

ST2 concentrations were determined as described in Example 1, above, in a population of 350 patients who presented to the ED with chest pain. Serum samples were obtained and ST2 measurements made at baseline, and 90 and 180 minutes later for most patients. Also for most patients, the baseline sample was collected within 2 hours of onset of symptoms.

17 patients had final diagnosis of MI, and 5 of these had ST2≧0.23 (0.25-0.65). Two of these patients were troponin negative. 11 patients had very high ST2 levels (0.97-9.22), but none of these patients had confirmed final diagnosis of MI and were all troponin negative, though all had severe diseases, including COPD, lymphoma, sepsis, alcohol abuse, and pulmonary embolism. The ST2 levels and diagnoses for these 11 patients are shown in Table 3; ST2 1 is the baseline level, ST2 2 is 90 minutes later, and ST2 3 is at 180 minutes.

TABLE 3
Non-MI Patients with High ST2 Levels

| | ST2 1 | ST2 2 | ST2 3 | |
|---|---|---|---|---|
| | (baseline) | (90 mins) | (180 Mins) | |
| Patient | (ng/ml) | (ng/ml) | (ng/ml) | Final Diagnosis |
| 811 | 1.43 | 1.62 | 1.63 | COPD with heart failure following |
| | | | | coronary artery bypass graft surgery and |
| | | | | pulmonary hypertension |
| 847 | 2.37 | 4.44 | 3.53 | Pulmonary embolism |
| 873 | 2.36 | 2.42 | 2.74 | Reactive airway disease (RAD) |
| 898 | 1.32 | 1.24 | 1.66 | History of heart failure following |
| | | | | coronary artery bypass surgery |
| 920 | 6.03 | 9.22 | | Bacteremia sepsis |
| 928 | 3.80 | 4.69 | 3.99 | Hypertension and alcohol abuse |
| 952 | 6.76 | | | Alcohol abuse, gastritis and pulmonary |
| | | | | hypertension |
| 953 | 3.77 | | | History of heart failure following |
| | | | | coronary artery bypass surgery |
| 1055 | 1.42 | 1.28 | 1.13 | Upper respiratory infection (URI) |
| 1213 | 0.97 | 1.19 | 1.07 | Pulmonary embolism and pericarditis |
| 1245 | 4.11 | 6.46 | | Lymphoma and hypertension |
| 1280 | 1.30 | 1.33 | | COPD |

These results demonstrate that the presence of elevated ST2 (e.g., above 0.2 ng/ml) in patients with chest pain who are troponin negative is associated with a high probability of pulmonary disease.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A kit for determining the likelihood of the presence of cardiovascular disease (CVD) or pulmonary disease (PD) in a subject, the kit comprising (i) one or both of a binding composition that specifically binds to IL-33 and a binding composition that specifically binds to ST2, (ii) a binding composition that specifically binds to a biomarker for CVD, and (iii) instructions for use in a method of determining the likelihood of the presence of CVD or PD in a subject, the method comprising:
determining a level of one or both of IL-33 and ST2 in a sample from a subject;
determining a level of a biomarker for CVD in the sample; and
comparing the level of IL-33 or ST-2, and the level of the CVD biomarker, in the sample to preselected corresponding reference levels;
wherein the levels in the sample as compared to the reference levels is correlated with the likelihood of the presence of CVD or PD.

2. The kit of claim 1, wherein the IL-33 and/or ST2 and the CVD biomarker comprise polypeptides, and the binding composition comprises an antibody or antigen binding fragment thereof that binds specifically to the polypeptides.

3. The kit of claim 1, wherein the IL-33 and/or ST2 and the CVD biomarker comprise nucleic acids and the binding composition comprises a probe that binds specifically to nucleic acid.

4. The kit of any of claims 1 to 3, wherein the CVD biomarker is a natriuretic peptide selected from the group consisting of brain natriuretic peptide (BNP), prohormone BNP (proBNP), N-terminal proBNP (NT-proBNP), atrial natriuretic peptide (ANP), proANP, or NT-proANP.

5. A method of determining the likelihood of the presence of cardiovascular disease (CVD) or pulmonary disease (PD) in a subject, the method comprising:
determining a level of a first biomarker selected from the group consisting of IL-33 and ST2 in a sample from a subject;
determining a level of a second biomarker for CVD in the sample; and
comparing the level of the first biomarker and second CVD biomarker in the sample to preselected corresponding reference levels;
wherein the levels of the biomarkers in the sample as compared to the reference levels is correlated with the likelihood of the presence of CVD or PD.

6. The method of claim 5, wherein the subject has chest pain or dyspnea.

7. The method of claim 5, wherein the CVD biomarker is a natriuretic peptide selected from the group consisting of brain natriuretic peptide (BNP), prohormone BNP (proBNP), N-terminal proBNP (NT-proBNP), atrial natriuretic peptide (ANP), proANP, or NT-proANP.

8. The method of claim 5, wherein determining a level of IL-33 in the sample comprises determining a level of one, two, or all three of mature IL-33, pre-IL-33, and pro-IL-33.

9. The method of claim 5, wherein the sample comprises blood, serum, plasma, urine, or body tissue.

10. The method of claim 5, wherein the reference levels represent a level in a subject who has a high likelihood of having pulmonary disease.

11. The method of claim 5, wherein the reference levels represent a level in a subject who has a high likelihood of having cardiovascular disease.

12. The method of claim 5, wherein the reference levels represent a level in a subject with a known severity of cardiovascular or pulmonary disease.

13. The method of claim 5, wherein determining a level of a biomarker in the sample comprises contacting a binding composition to the sample, wherein the binding composition specifically binds to the biomarker, and measuring or determining the specific binding of the binding composition to the sample.

14. The method of claim 13, wherein the binding composition comprises an antibody that binds specifically to a biomarker comprising a polypeptide.

15. The method of claim 13, wherein the binding composition comprises an oligonucleotide probe that bind specifically to a biomarker comprising a polynucleotide.

16. The kit of any of claims 1-3 or the method of claim 5, wherein the CVD is acute coronary syndrome (ACS), myocardial infarction, heart failure, angina, cardiac hypertrophy, arteriosclerosis, myocarditis, pericarditis, endocarditis, or stroke.

17. The kit of any of claims 1-3 or the method of claim 5, wherein the PD is chronic obstructive pulmonary disease (COPD), asthma, pneumonia, pneumothorax, pulmonary embolism, pleural effusion, metastatic disease, pulmonary edema, gastrooesophageal reflux disease with aspiration, pulmonary embolism, and restrictive lung disease.

18. The method of claim 5, further comprising determining a level in the sample of each of one or more other biomarkers.

19. The method of claim 18, wherein the one or more other biomarkers are selected from the group consisting of troponin, creatine kinase MB (CK-MB), ischemia-modified albumin (IMA), Interleukin-6 (IL-6), C-reactive protein (CRP), creatinine, D-dimers, blood urea nitrogen (BUN), liver function enzymes, albumin, and bacterial endotoxin.
